(19) United States
(12) Patent Application Publication
(10) Pub. No.: US 2024/0139019 A1
(43) Pub. Date: May 2, 2024

(54) External Nasal Dilator

(71) Applicant: Foundation Consumer Brands, LLC, Wilmington, DE (US)

(72) Inventors: Joseph A. Matthias, Andover, NJ (US); Yu Y. Shao, King of Prussia, PA (US); Snigdha Mishra, Parsippany, NJ (US)

(21) Appl. No.: 18/479,509

(22) Filed: Oct. 2, 2023

Related U.S. Application Data
(63) Continuation of application No. 12/771,431, filed on Apr. 30, 2010, now abandoned.
(60) Provisional application No. 61/174,669, filed on May 1, 2009.

(51) Int. Cl. A61F 5/08

(52) U.S. Cl. CPC A61F 5/08

(57) ABSTRACT

A nasal dilator with improved functionality by virtue of a resilient element comprised of at least three resilient bands, a first outer resilient band that is secured to run along the length of the nasal dilator truss member, a second outer resilient band that is spaced apart from the first resilient band and is secured to run along the length of the nasal dilator truss member and at least one intermediate resilient band positioned between said first and second outer resilient bands that is spaced apart from both first and second outer resilient bands and is also secured to run along the length of the nasal dilator truss member. The intermediate resilient band or bands alters the force vector characteristics of the nasal strip as a whole, thereby providing targeted spring force to a more concentrated area of the nose when in use.

### FIELD OF THE INVENTION

The present invention relates to an external nasal dilator, more specifically to an improved external nasal dilator, which provides a focused and efficient spring force to the outer wall tissues of the first and second nasal passages.

### BACKGROUND OF THE INVENTION

Nasal dilators, both internal and external, which act on the outer wall tissues of the nasal passages are well known. For example, external nasal dilators are disclosed in U.S. Pat. Nos. 5,533,499, 5,533,503 and 6,318,362 to Johnson. These nasal dilators comprise a truss member having a first end region adapted to engage the outer wall tissues of a first nasal passage and a second end region adapted to engage the outer wall tissues of a second nasal passage. The first and second end regions are coupled to one another by an intermediate segment. The intermediate segment is configured to traverse a portion of the nose located between the first and second nasal passages, A resilient element or spring member extends along the length of the truss member. The spring member, when the truss is secured in place, acts to stabilize the outer wall tissue and thereby prevent the outer wall tissues of the first and second nasal passages from drawing in during breathing.

In one known nasal dilator embodiment, such as disclosed in U.S. Pat. No. 6,318,362, the resilient element may consist of a pair of resilient bands. The first resilient band is secured to run along the length of the nasal dilator truss member. The second resilient band is spaced apart from the first resilient band and is also secured to run along the length of the nasal dilator truss member. The first and second bands are relatively stiff and are oriented generally parallel to one another and substantially parallel to the longitudinal extent of the nasal dilator. The resiliency of the first and second bands prevents the outer wall tissues of the first and second nasal passages from drawing in during breathing.

In another known nasal dilator embodiment, such as disclosed in US Patent Application Publication 2005/0081857, the resilient element may be comprised of a plurality of small filaments for keeping the nasal passages from drawing in during breathing. The filaments may be a variety of shapes and sizes and may run both along the length of the nasal dilator and at a variety of different angles relative to the length of the nasal dilator. These filaments allow the nasal dilator to be removed from the nose in a top to bottom fashion. The multiple filament construction allows for a greater peel angle than seen in earlier nasal dilator embodiments and thereby results in less peel force being transferred to the nose.

While known nasal dilator embodiments are efficacious for many, there is significant variation in the size and structure of individual noses and, it has been found that certain noses are not wholly responsive to traditional nasal dilators, such as those sold under the tradename Breathe Right® by GlaxoSniithKline Consumer Healthcare. Thus, there exists a need to develop nasal dilators that can provide an increased and targeted spring force, which will be efficacious for a greater variety of noses, particularly for those noses that do not respond to those nasal dilators currently commercially available.

### SUMMARY OF THE INVENTION

The present invention includes an external nasal dilator with improved functionality by virtue of a resilient element comprised of at least three resilient bands. The nasal dilators of the present invention comprise a first outer resilient band that is secured to run along the length of the nasal dilator truss member. A second outer resilient band is spaced apart from the first resilient band and is also secured to run along the length of the nasal dilator truss member. Positioned between the first and second outer resilient bands is at least one intermediate resilient band that is spaced apart from both first and second outer resilient bands and is also secured to run along the length of the nasal dilator truss member. The intermediate resilient band or bands alters the force vector characteristics of the nasal strip as a whole, thereby providing targeted spring force to a more concentrated area of the nose when in use. This provides a more efficacious nasal dilator, as determined by a change in minimum cross sectional area (MCA) of the nasal valve region and nasal volume in the anterior nasal cavity (0-3 cm beyond the nostril) within a certain range of nose widths as measured across from alar crease to another.

In one embodiment, three resilient bands are secured to run along the length of the nasal dilator truss member and are oriented generally parallel to one another and substantially parallel to the longitudinal extent of the nasal dilator. In another embodiment, the overall length of the resilient element can be varied, in order to achieve improved efficacy for nasal widths that fall outside the range of nasal widths that generally respond to currently available nasal dilator products.

### BRIEF DESCRIPTION OF THE DRAWINGS

FIG. 1 is a perspective view of one embodiment of the nasal dilator of the present invention.

FIG. 2 is a top view of one embodiment of the nasal dilator of the present invention.

FIG. 3 is a bottom view of one embodiment of nasal dilator of present invention.

FIG. 4 is a side elevational view of one embodiment of the nasal dilator of the present invention.

FIG. 5 is an end elevational view of one embodiment of the nasal dilator of the present invention.

FIG. 6 is a perspective view of a second embodiment of the invention.

FIG. 7 is a top view of the second embodiment of the invention

FIG. 8. is a perspective of a third embodiment of the invention.

FIG. 9 is a top view of the third embodiment of the invention.

FIG. 10 is a photographic comparative end view of a traditional dual band nasal dilator and one embodiment of the nasal dilator of the present invention, wherein both nasal dilators are attached to a dowel representing the side of a human nose.

FIG. 11 is a photographic comparative end view of a traditional dual resilient band nasal dilator and one embodiment of the nasal dilator of the present invention.

FIG. 12 is a photographic side view of one embodiment of the present invention with force vectors represented by the arrows; the red arrow depicts the force vector of the outer resilient bands, the blue arrow depicts the force vector of the intermediate resilient band,

FIG. 13 is a graphical depiction of the effect of a traditional dual band nasal dilator versus a tri band embodiment of the present invention on nasal volume across a variety of nose widths.

FIG. 14 is a depiction of the nose width measurement.

FIG. 15 shows scheme 1.

FIG. 16 shows scheme 2.

FIG. 17 shows scheme 3.

FIG. 18 shows scheme 4.

FIG. 19 shows scheme 5.

FIG. 20 shows scheme 6.

FIG. 21 shows scheme 7.

FIG. 22 shows Table 1: treatment comparisons between three-band external dilator and marketed, two-band BRNS by acoustic rhinometry.

FIG. 23 shows Table 2: MCA and Volume change from baseline in 7 non-responders to current BRNS.

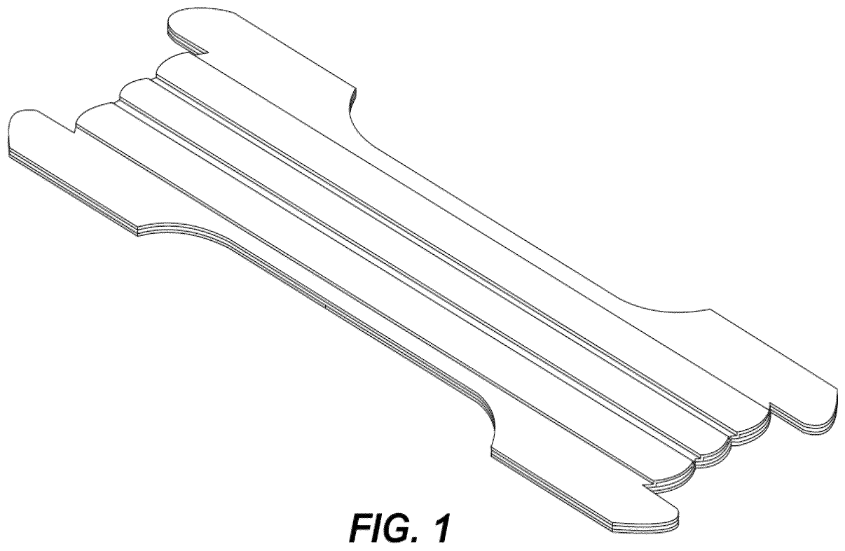

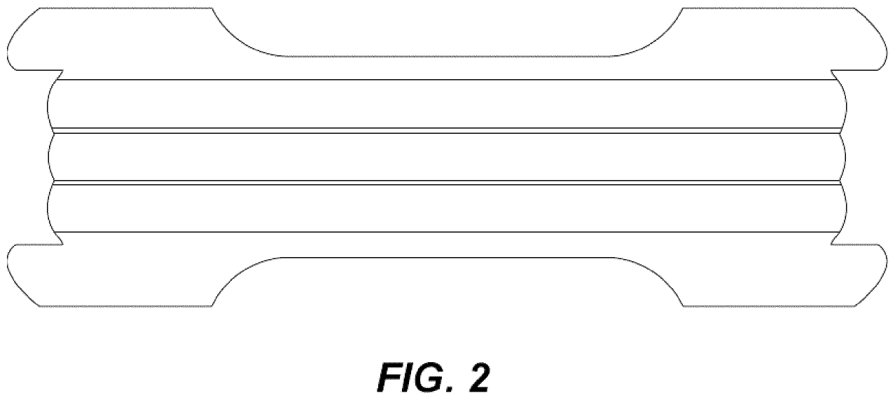

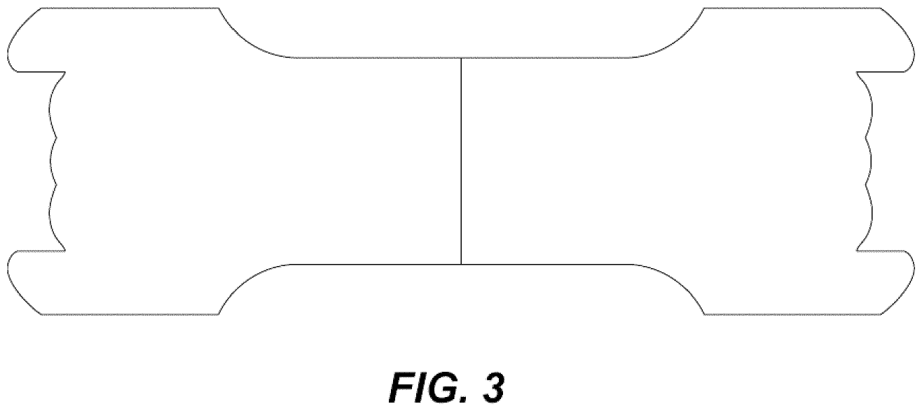

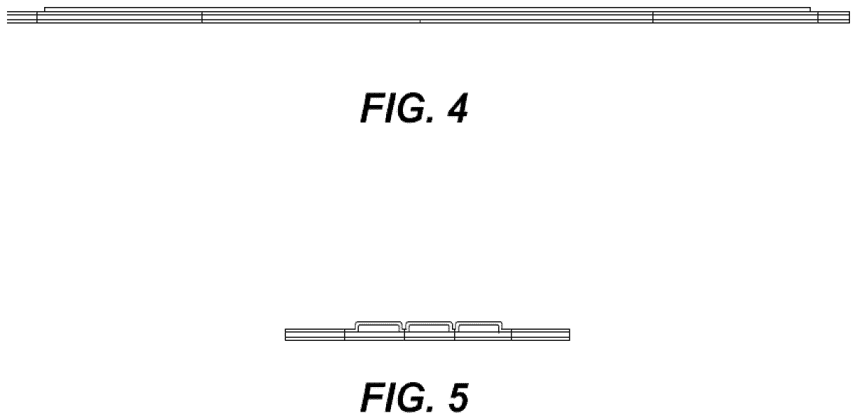

### DETAILED DESCRIPTION OF THE INVENTION

All publications, including but not limited to patents and patent applications, cited in this specification are incorporated herein by reference as though fully set forth.

Unless otherwise stated, as used herein, the modifier “a” includes one or more of the components modified. The present invention may comprise, consist essentially of, or consist of the components set forth below, unless otherwise stated.

The present invention relates to an improved external nasal dilator with improved functionality by virtue of a resilient element comprised of at least three resilient bands acting in combination to open the nasal valve, decrease nasal resistance and improve pulmonary effort during breathing.

The nasal dilators of the present invention comprise a truss member including a flexible strip of base material and a resilient element. The truss defines a first end region adapted to engage the outer wall tissues of a first nasal passage and a second end region adapted to engage the outer wall tissues of a second nasal passage. An intermediate segment connects the first and second end regions and is configured to traverse the bridge of the nose located between the first and second nasal passages. The flexible strip of base material may be formed of any material that allows the skin of the nose to breathe, to maximize comfort and minimize irritation. The flexible strip of material can be any thin, flexible, breathable material for maximizing comfort. Preferably, this material permits the passage of air and moisture. In one embodiment, the flexible strip of base material is a strip of interwoven or non-woven strip of polyester fabric. For example, a suitable nonwoven spun-laced 100% polyester fabric for use as the flexible strip of material in the present invention is available under the tradename Sontara® from E.I. DuPont Nemours & Co. Alternatively, the flexible strip of base material may be formed of a plastic film material. While the plastic film material may not be breathable, permitting passage of air and moisture, it may have comfort benefits of being easy to remove after a period of use.

The truss member may further include a flexible strip of backing material so that the resilient element is disposed between the layer of backing material and the flexible strip of base material. The flexible strip of backing material may be made of rubber, vinyl, cloth, soft plastic or any other material known in the art to be pliable under the conditions for which the nasal dilator is to be used.

An adhesive material may be placed on one side of the truss member such that the nasal dilator may be removably affixed to the nose of a user. In one embodiment the adhesive material is placed on a first side of the truss member and the resilient element is placed on a second side of the truss member. In another embodiment the adhesive material and resilient element are both placed on a first side of the truss member. In another embodiment, the adhesive material is a pressure sensitive biocompatible adhesive that is compatible with the skin of the user but strong enough that it can maintain the nasal dilator in the correct position during use. Suitable adhesives for use in the present invention include, but are not limited to, solvent or water-based pressure-sensitive adhesives, such as acrylate adhesives, thermoplastics “hot melt” adhesives, double-sided adhesive tapes, elastomer-based adhesives and acrylic adhesives. Optionally a release liner may be used to protect the pressure sensitive adhesive in transit and prior to use which can be readily removed from the adhesive material.

Those of skill in the art will recognize that all of the materials used to make the truss member must withstand the forces placed thereon and will also withstand the foreign objects and materials that the nasal dilator will come in contact with, i.e. water, sweat, skin oils, etc.

The resilient element is fixedly attached to or integrated within the truss member. The resilient element comprises a first outer resilient band that is secured to run along the length of the nasal dilator truss member, a second outer resilient band that is spaced apart from the first resilient band and is also secured to run along the length of the nasal dilator truss member and positioned between the first and second outer resilient bands at least one intermediate resilient band that is spaced apart from both first and second outer resilient bands and is also secured to run along the length of the nasal dilator truss member. In one embodiment, the resilient bands are individually adhesively secured within the truss member between the layer of backing material and the flexible strip of base material. In other embodiments where no backing layer is present, each resilient band may be adhesively attached to the flexible strip of base material.

Each of the resilient bands that make up the resilient element have a width to thickness ratio of from about 1:1 to about 15:1, i.e. the resilient bands are substantially square or rectangular in cross sectional shape. Each of the resilient bands is initially coplanar. The total spring force delivered by the resilient element as a whole should be from about 15 grams (gm) to about 60 gm. In one embodiment, the total spring force delivered by the resilient element as a whole should be from 25 gm to about 35 gm.

The total number of resilient bands may vary but should not total more than seven resilient bands in any one resilient element. Preferably, the resilient element will comprise from 3 to 5 resilient bands, more preferably the resilient element comprises 3 resilient bands. The length of the resilient bands may be the same or of similar length or the lengths of the bands of the resilient element may vary. In one embodiment of the invention, all of the bands in the resilient element are the same length. In another embodiment of the invention, all of the resilient bands are similar in length and differ by no more than 20% at any given time. In another embodiment of the invention, the resilient bands are of different lengths such that they differ 20-40% or 20-35% in length. In another embodiment of the invention, the outer two resilient bands are the same or similar in length and the intermediate band(s) are 20-40% or 20-35% shorter than the outer bands.

In another embodiment of the invention, the resilient element comprises three resilient bands wherein the three resilient bands are all of equal length. In another embodiment, the resilient element comprises three resilient bands wherein the three resilient bands are similar in length such that they differ by no more than 20% at any given time. In another embodiment of the invention, the two outer resilient bands are the same length and the one intermediate resilient band is 20-40%, preferably 20-35%, more preferably 20-25% shorter than the outer two resilient bands.

In another embodiment of the present invention, the resilient element comprises three resilient bands, wherein one of the outer resilient bands is 15-40%, preferably 15-30%, more preferably 15-20% shorter than the other two resilient bands. The other two resilient bands, meaning the second outer band and the one intermediate band, are the same length. The resilient element is configured such that when the nasal dilator is worn, the shorter outer resilient band is above the other two resilient bands such that the shorter resilient band is closest to the forehead of the user.

The resilient bands may be formed of a variety of polymeric materials and other materials that have a tendency to return to a normally planar state upon the removal of an external bending force. For example, an industrial grade biaxially oriented polyester about 0.010 inches in thickness and from about 0.060 inches to about 0.150 inches in width is suitable for use in the present invention. Using a polymeric material that is relatively thin, as just described for each of the resilient bands, enhances the axial, torsional flexibility of each of these bands about the longitudinal extent of each, depending on the width of the actual bands used.

The overall length of the resilient element is from about 40 mm to about 70 mm. In one embodiment the overall length of the resilient element is from about 40 mm to about 65 mm. In another embodiment the overall length of the resilient element is from about 50 mm to about 60 mm.

The resiliency of the resilient element and the tendency of the resilient bands to return to their normally planar state once having the ends thereof forced toward one another provides an outward pull on the outer wall tissues of the nasal passages of the user when the nasal dilator is properly positioned on the nose of the user. This outward pull opens the nasal valve, decreases nasal resistance and improves pulmonary effort during breathing. The flexibility of the truss member, the resiliency of the resilient element, and the relatively slight overall thickness of the nasal dilator all allow the nasal dilator to conform closely about the curves of the nose of each individual user and in turn increases the comfort of the nasal dilator to the user.

The force imparted by the nasal dilators of the present invention was measured using a standard QC spring force test wherein the nasal dilator (or strip) is compressed lengthwise to a determined distance, from a starting distance of 1.45 inches to a distance of 1.20 inches. This distance approximates the distance the ends of the nasal strip would be spread apart on a human nose when in use. An Instron strain gauge is used to measure the pressure exerted against the load cell. The force measured at this final position is the spring force, typically reported in grams. In one embodiment, where the resilient element is comprised of three resilient bands, approximately 32 grams of force are delivered.

Airflow resistance provided by the nose during breathing is essential for preconditioning of the inspired air that is required to promote healthy pulmonary function. In healthy individuals, nasal resistance provides almost two thirds of the total airway resistance and most of this resistance occurs in the anterior 2-3 cm of the nose, in the region known as the nasal valve, Most of the pulmonary effort in the normal, healthy population is consumed to overcome this resistance.

The nasal valve is usually the narrowest part of the nose and is a roughly triangular opening in the anterior portion of the nasal airway formed by the nasal septum, the caudal border of the upper lateral cartilage, the head of the inferior turbinate, and the pyriform aperture and the tissues surrounding it. In those individuals with higher than normal nasal resistance, an external nasal dilator can be utilized to increase the cross sectional area of the nasal valve, decreasing airway resistance, and normalizing pulmonary effort.

Acoustic rhinometry is a diagnostic technique used to assess internal nasal anatomy through analysis of the strength and timing of reflections of a sound pulse introduced via the nostrils. The technique is rapid, reproducible, non-invasive, and requires minimal cooperation from the subject. Through this technique, a graph of nasal cross-sectional area as a function of distance from the nostril is produced, from which the MCA and nasal volume of the nasal cavity can be derived. The minimum cross sectional area (MCA) is the narrowest constriction of the nasal passage found in the anterior 0 to 3 cm portion of the nasal airway. The MCA is a measure of the airway opening within the nasal valve, and is a two-dimensional measure, Nasal volume of the anterior nasal cavity (nasal volume) is the summation of the two-dimensional measurements from the anterior 0 to 3 cm portion of the nasal passage. In conjunction with the MCA, the nasal volume provides an additional measure of the passage in the nasal valve area, and is a 3-dimensional measure.

A nasal patency study was conducted in 82 normal healthy individuals. The primary objective of this study was to compare the effect of a three band embodiment of the current invention versus the marketed dual band BreatheRight® nasal dilator product on the minimum cross sectional area (MCA) and nasal volume of the nose using acoustic rhinometry.

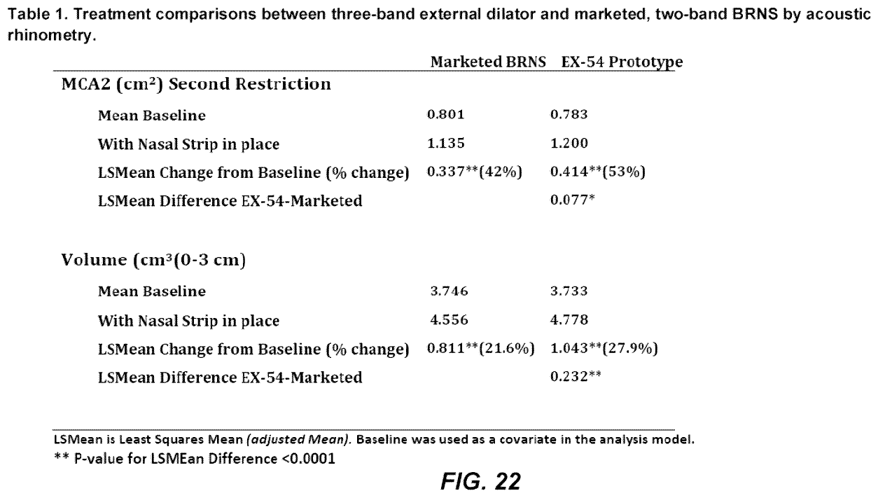

This was a single center, randomized, single blind, cross-over study of a prototype dilator (or strip) (EX-54-3 springs, each 54 mm in length) vs. marketed Breathe Right® nasal dilator (Tan). The primary nose width was measured across from one alar crease of the nose to the other. The three band nasal dilator demonstrated enhanced effect on measurements of MCA and nasal volume, as determined by acoustic rhinometry, when compared to the control (FIG. 22)

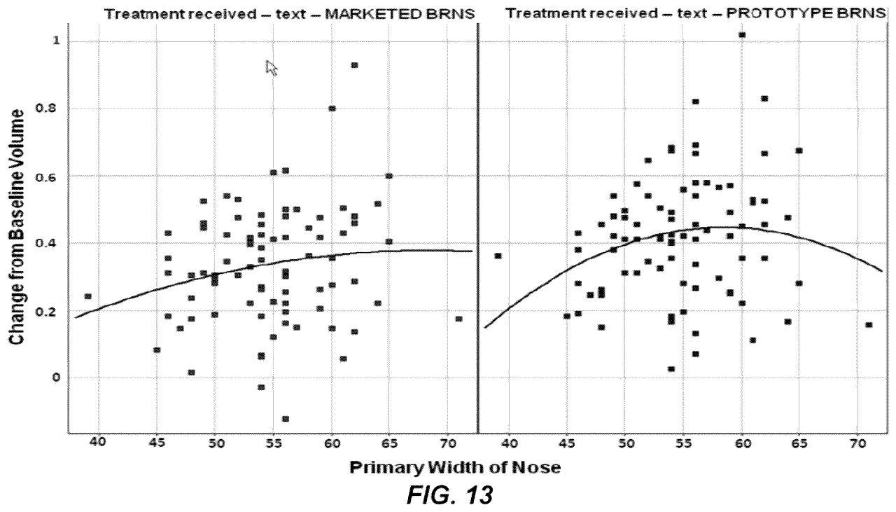

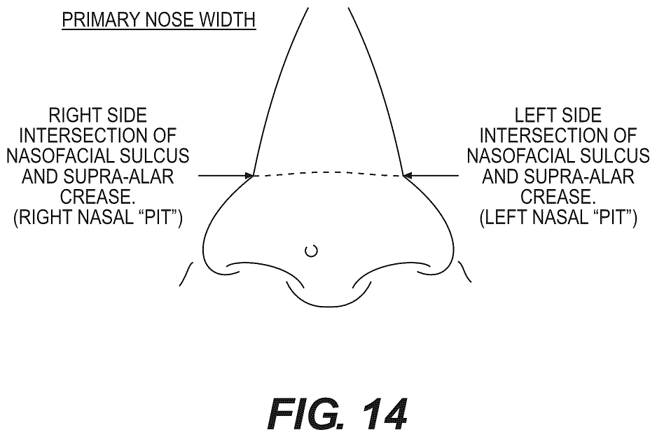

A surprising factor in the enhanced effect was the greater response to the three band nasal dilator over a narrower range of nose widths. Specifically, subjects with a nose width in the range of 50-65 mm showed a greater response to the three band dilator than to the control dilator. Subjects with a nose width in the narrower (<50 mm) and broader (>65 mm) range showed a dramatic reduction in efficacy compared to control as depicted in FIG. 13.

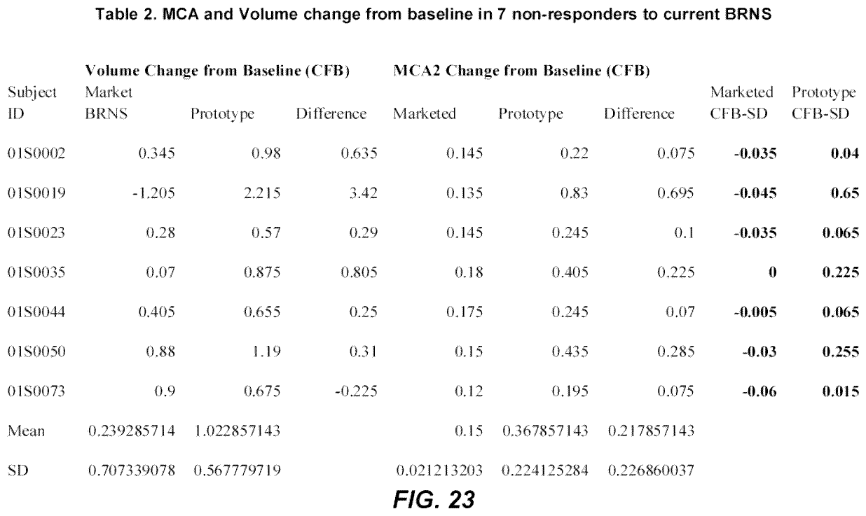

Moreover, seven subjects enrolled in this study (approximately 10%) did not exhibit any measurable change from baseline for MCA and nasal volume using the control two-band dilator. These non-responders demonstrated an increase in both MCA and nasal volume upon treatment with the three-band dilator (FIG. 23). Consequently, the three-band strip was able to demonstrate a dilation effect on the nasal valve of these seven subjects.

A second nasal patency study was conducted in 30 normal healthy subjects. The primary objective of this study was to assess the effect of several of the embodiments of the current invention in comparison to the marketed dual band Breathe Right® nasal dilator (Tan) on the minimum cross sectional area (MCA) and nasal volume using an acoustic rhinometer.

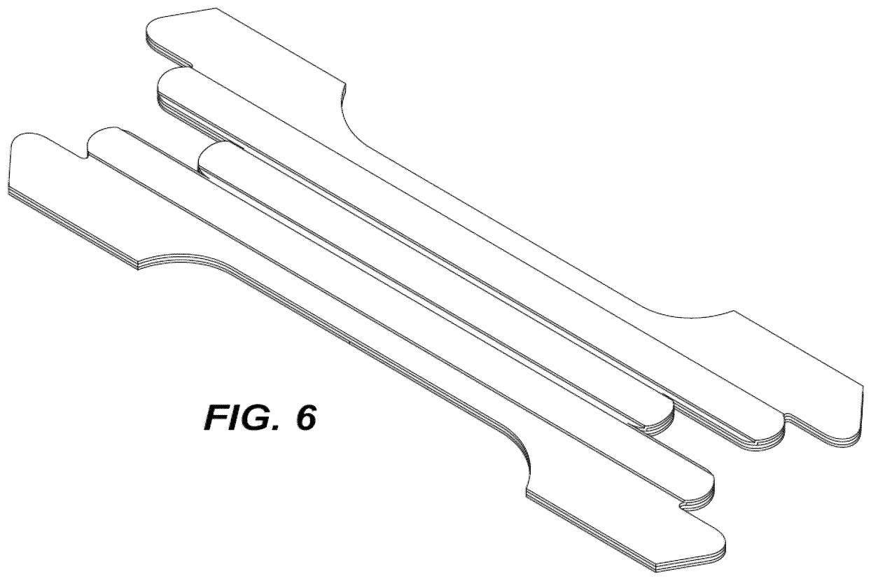

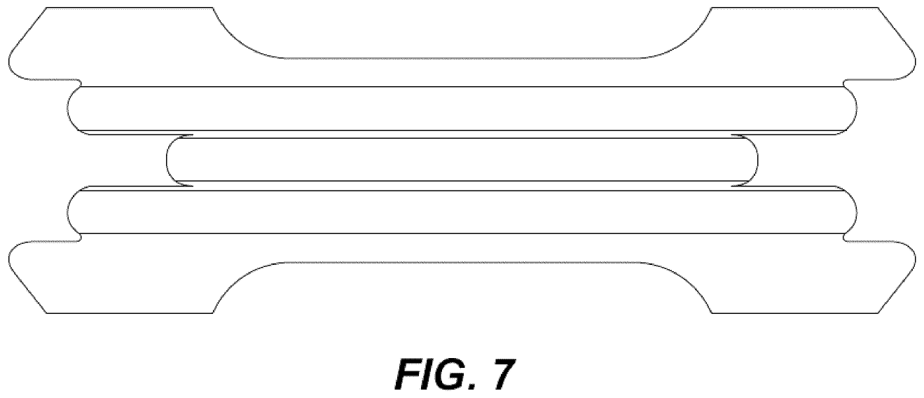

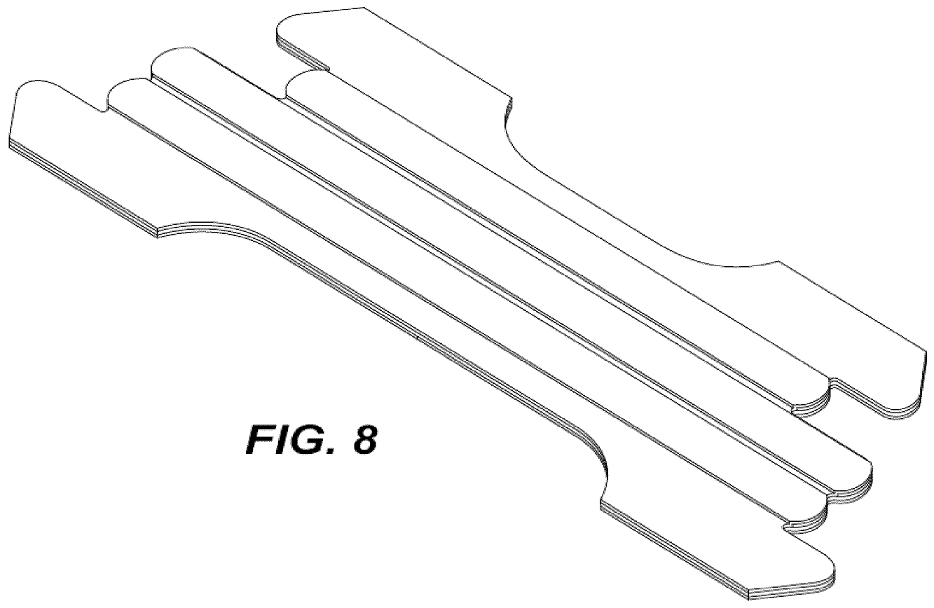

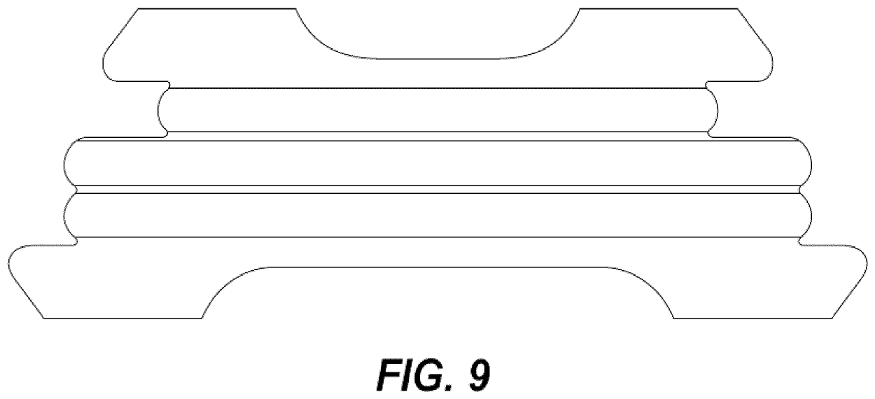

This was a single center, randomized, cross-over study. In comparison to the currently marketed dual band Breathe Right® nasal dilator (Tan) (control), two of the embodiments showed improvements in both MCA and volume. The embodiment as depicted in FIGS. 6 and 7 wherein the two outer resilient bands are 57.5 mm in length and the intermediate band is 43.1 mm in length, and wherein the widths of all the bands are about 3.2 mm, showed a 48.6% increase in MCA (vs 33.9% increase for the control) and a 20.4% increase in volume (vs 16.3% increase for the control) beyond baseline. The embodiment as depicted in FIGS. 8 and 9 wherein the first outer resilient band (i.e. the top band) is 43.7 mm in length and the second outer and intermediate bands are 57.5 mm in length and wherein the widths of all the bands are about 3.2 mm, showed a 38.9% increase in MCA (vs 33.9% increase for the control) and a 20.0% increase in volume (vs 16.3% increase for the control) beyond baseline. These data indicate that the spring length, number and position play important roles in the performance of the strip.

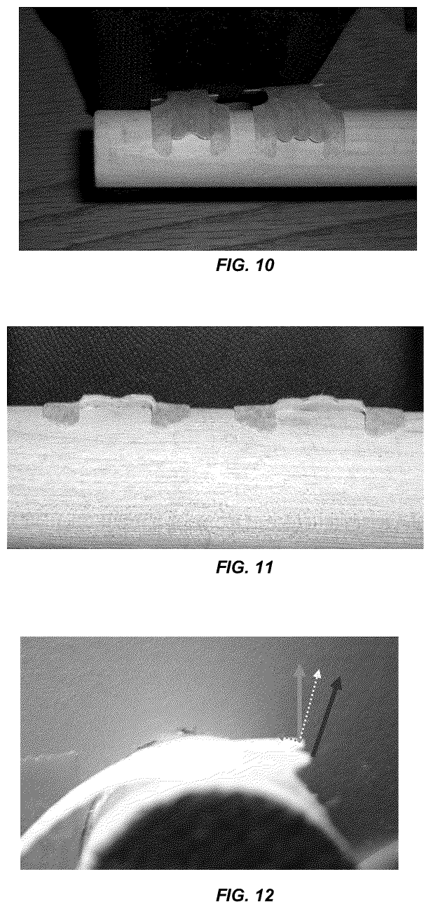

FIGS. 10 and 11, are photographic depictions of a three band nasal dilator of the present invention (EX-54-3 springs, each 54 mm in length) and the two band control. It can be seen that the end of the intermediate resilient band is in a different position relative to the ends of the two outer resilient bands when in place on the nose. From this higher position the intermediate resilient band is likely able to exert significantly more outer nose tissue distending force on certain types of noses. In this case, the noses most benefiting from the three band nasal dilator embodiment appear to be in the range of about 50 mm to about 60 mm nose widths.

The intermediate resilient band would appear to change the force vector characteristics of the nasal strip, FIG. 12 is a photographic side view of a three band nasal dilator of the present invention (EX-54-3 springs, each 54 mm in length) showing that the two outer resilient bands still act in a general orthogonal (to the surface of the nose) direction which is indicated by the solid red arrow. It is believed that the intermediate resilient band is acting in the more upwards direction toward the top of the nose (parallel to the surface of the nose) defined by the solid blue arrow. Both outward (dotted yellow arrow or solid red arrow) and upward (dotted green arrow) movement of the outer side of the nose will dilate the nasal valve. It is further believed that the vector of the intermediate resilient band can be broken down into the portion acting orthogonally (dotted yellow arrow) like the two outer resilient bands and the portion acting in a parallel direction (dotted green arrow). Both dual band dilators and the three band dilator embodiment have outward/orthogonal force vectors but only the three band nasal dilator embodiment appears to have significant upward vectors.

### Analysis of Forces in the Two Spring Design:

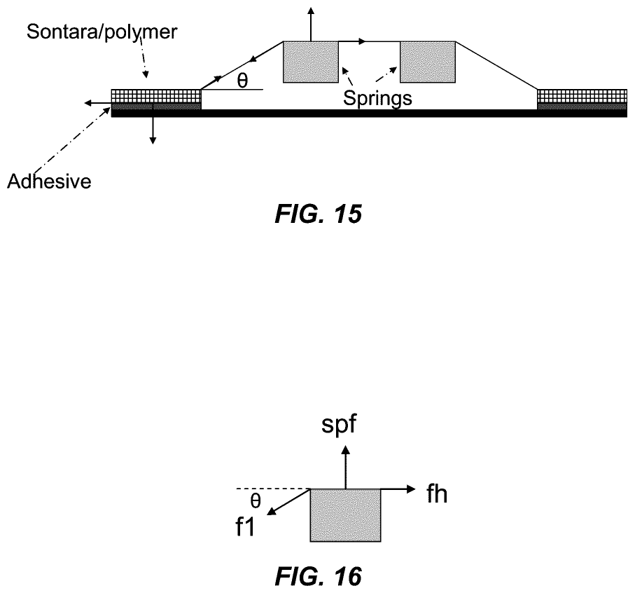

Scheme 1 (FIG. 15) represents the cross sectional area of a two spring design with forces shown.

The two springs cause the lifting action force spf that is balanced by forces 11 and fh, as shown in Scheme 2 (FIG. 16) wherein spf=f1 sin θ and f1 cos θ=fh.

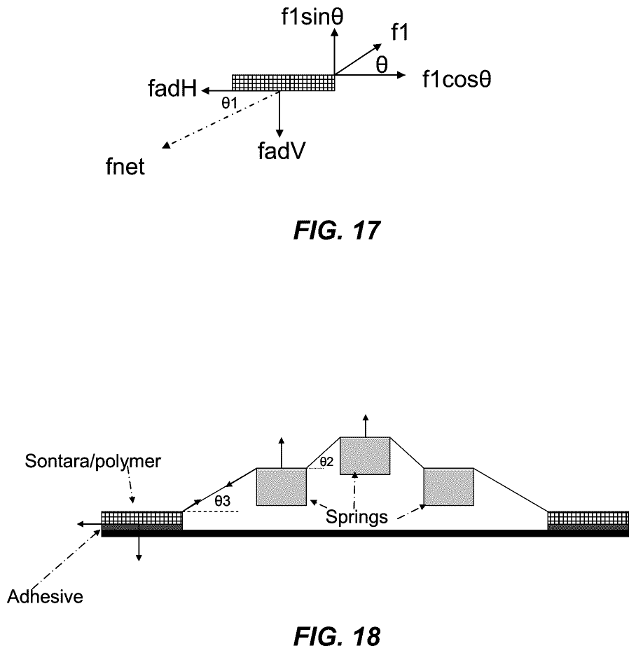

The force f1 then transforms to the forces on the adhesive fades and fadV that cause a lifting action as shown in Scheme 3 (FIG. 17), wherein f1 sin θ=fadV and f1 cos θ=fdH. The direction 81 of the net lifting force fnet can be found by: θ1=tan⁻¹(fadV/fadH).

### Analysis of Forces in the Three Spring Design:

Scheme 4 (FIG. 18) represents the cross sectional area of a three spring design with forces shown.

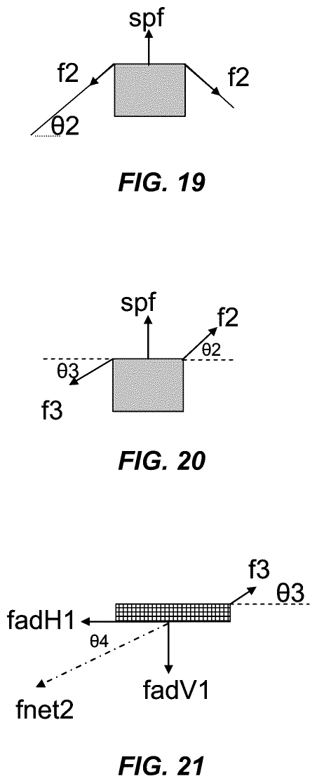

The central spring causes a lifting action force spf that is balanced by forces f2 as shown in Scheme 5 (FIG. 19), wherein spf=2 f2 cos θ2. The force f2 can be obtained by the known parameters θ2 and the spring force spf. The force f2 then transforms to a force f3, as shown in Scheme 6 (FIG. 20), wherein 12*cos θ2=f3*cos θ3 and spf f2*sin θ2=f3*sin θ3, that can be assumed to be acting at approximately the same angle as in the two spring design.

The force f3 then transforms itself to the new lifting forces on the adhesive fadH1 and fadV1 that cause a lifting action, as shown in Scheme 7 (FIG. 21), wherein f3*cos θ3=fadH1 and f3*sin θ3=fadV1. The direction θ4 of the net lifting force fnet can be found by: θ4=tan⁻¹ (fadV1/fadH1).

The above analysis shows that the force f3 is used to balance two upwards forces spf and f2, as compared to the force f1 in the two spring design that is only used to balance one force spf. This will result in the force f3 being higher than 11. Also, the angle θ3 is going to be larger than 0 because the middle spring will cause the side two springs to lift more. This will cause the new net lift angle θ4 to be higher than the old net lift angle θ1 for the two-spring design.

It should be understood that slight variations in the total length of the resilient element could be used to target noses outside of the current 50 to 60 mm range. For example, a shorter resilient element may be useful for treating noses that are less than about 50 mm in width. A slightly longer resilient element may be useful for treating noses that are greater than about 60 mm in width. Further, the incorporation of additional resilient bands may further focus the spring force applied to the nose to achieve even great response for a more narrow range of nose widths.

1-11. (canceled)

12. An external nasal dilator configured to be connected to a nose, the external nasal dilator comprising:
a flexible base comprising:
a first end region configured to engage outer wall tissue of a first nasal passage of the nose;
a second end region configured to engage outer wall tissue of a second nasal passage of the nose;
an intermediate region connecting the first end region and the second end region configured to transverse a bridge of the nose;
a resilient element fixedly attached to or integrated within the flexible base, wherein the resilient element comprises:
a first outer resilient band that extends between the first end region and the second end region of the flexible base,
a second outer resilient band that is spaced apart from the first outer resilient band and extends between the first end region and the second end region of the flexible base;
an intermediate resilient band that is positioned between the first outer resilient band and the second outer resilient band and extends between the first end region and the second end region, which is configured to exert forces on the first outer resilient band and the second outer resilient band causing the resilient element to transition between an unbiased, planar position, when the first end region and the second end region are not engaged to the nose, and a biased, non-planer position, in which the first and second outer resilient bands move outwardly by a first distance and the intermediate resilient band moves outwardly by a second distance, which is greater than the first distance; and
an adhesive over the first end region, the second end region, and the intermediate region of the flexible base for removably affixing the external nasal dilator to the nose.

13. The external nasal dilator of claim 12, further comprising a layer of backing material connected to the flexible base and enclosing the resilient element between a surface of the flexible base and a surface of the layer of backing material.

14. The external nasal dilator of claim 13, wherein the layer of backing material comprises at least one of rubber, vinyl, cloth, plastic, or any combination thereof.

15. The external nasal dilator of claim 13, wherein the resilient bands are adhesively attached to the surface of the flexible base.

16. The external nasal dilator of claim 12, wherein the flexible base comprises a first side surface and a second side surface, and wherein the resilient element is fixed to one of the first side surface or the second side surface and the adhesive covers the other of the first side surface or the second side surface.

17. The external nasal dilator of claim 12, wherein the resilient element and the adhesive are on a same side surface of the flexible base.

18. The external nasal dilator of claim 12, wherein the resilient element is configured to return to the unbiased, planar position upon removal of the external nasal dilator from the nose.

19. The external nasal dilator of claim 12, wherein the flexible base comprises a strip of interwoven or non-woven strip of polyester fabric.

20. The external nasal dilator of claim 12, wherein the flexible base comprises a nonwoven spun-laced polyester fabric.

21. The external nasal dilator of claim 12, wherein each of the resilient bands of the resilient element has a width to thickness ratio of from about 1:1 to about 15:1.

22. The external nasal dilator of claim 12, wherein a total spring force delivered by the resilient bands of the resilient element is from about 15 grams to about 60 grams.

23. The external nasal dilator of claim 12, wherein the resilient bands comprise a biaxially oriented polyester that is about 0.060 inch to about 0.150 inch in width.

24. The external nasal dilator of claim 12, wherein an axial length of the intermediate resilient band is from 20% to 40% shorter than an axial length of the first outer resilient band or the second outer resilient band.

25. The external nasal dilator of claim 12, wherein an axial length of the first outer resilient band and the second outer resilient band differ by no more than 20%.

26. The external nasal dilator of claim 12, wherein an overall length of the resilient element is about 40 mm to about 70 mm.

27. The external nasal dilator of claim 12, wherein the adhesive comprises a solvent or water-based pressure-sensitive adhesive.

28. The external nasal dilator of claim 27, wherein the solvent or water-based pressure-sensitive adhesive comprises at least one of: an acrylate adhesive; a thermoplastic “hot melt” adhesive; a double-sided adhesive tape; an elastomer-based adhesive; an acrylic adhesive; or combinations thereof.

29. The external nasal dilator of claim 12, further comprising a release liner attached to the adhesive prior to use of the external nasal dilator, which is configured to be removed from the adhesive to attach the external nasal dilator to the nose.

30. The external nasal dilator of claim 12, wherein the intermediate resilient band is configured to exert a force on the nose in an upwards direction, which is substantially parallel to a surface of the nose.

31. The external nasal dilator of claim 30, wherein the first and second outer resilient bands are configured to exert forces on the nose in a direction that is substantially orthogonal to the surface of the nose and which is different from the upwards direction of the intermediate resilient band.
